(19) United States
(12) Patent Application Publication
(10) Pub. No.: US 2004/0152735 A1
(43) Pub. Date: Aug. 5, 2004

(54) NO-synthase inhibitors comprising N,N'-bis(2-pyridyl)methyl-N,N'-bis(3,4,5-trimethoxybenzyl) ethylenediamine

(75) Inventor: Marie-Madeleine Cals-Grierson, Meudon (FR)

Correspondence Address: BURNS DOANE SWECKER & MATHIS L L P, POST OFFICE BOX 1404, ALEXANDRIA, VA, 22313-1404, US

(73) Assignee: SOCIETE L'OREAL, S.A., PARIS (FR)

(21) Appl. No.: 10/733,387

(22) Filed: Dec. 12, 2003

Related U.S. Application Data
(63) Continuation of application No. PCT/FR02/02064, filed on Jun. 14, 2002.

(30) Foreign Application Priority Data
Jun. 15, 2001 (FR) 01/07878

(51) Int. Cl. A61K 31/444

(52) U.S. Cl. 514/332

(57) ABSTRACT

N,N′-bis(2-pyridyl)methyl-N,N′-bis(3,4,5-trimethoxybenzyl) ethylenediamine and therapeutic/cosmetic compositions comprised thereof are useful NO-synthase inhibitors, suited, inter alia, for slowing or even inhibiting cell differentiation and/or proliferation, and for inhibiting the degradation and/or destruction of cells.

### CROSS-REFERENCE TO PRIORITY/PCT APPLICATIONS

This application claims priority under 35 U.S.C. § 119 of FR 01/07878, filed Jun. 15, 2001, and is a continuation of PCT/FR 02/02064, filed Jun. 14, 2002 and designating the United States (published in the French language on Dec. 27, 2002 as WO 02/102344 A3; the title and abstract were also published in English), both hereby expressly incorporated by reference.

### CROSS-REFERENCE TO COMPANION APPLICATION

Copending application Ser. No. ______ [Attorney Docket No. 016800-654], filed concurrently herewith and assigned to the assignee hereof.

### BACKGROUND OF THE INVENTION

1. Technical Field of the Invention

The present invention relates to the use (therapeutic/cosmetic regime or regimen) of an effective amount of N,N′-bis(2-pyridyl)-methyl-N,N′-bis(3,4,5-trimethoxybenzyl)ethylenediamine, in a physiologically acceptable medium, in a composition or for the preparation of a composition, the N,N′-bis(2-pyridyl) methyl-N,N′-bis(3,4,5-trimethoxybenzyl)ethylenediamine or composition comprised thereof being useful for inhibiting NO-synthase.

2. Description of Background/Related/Prior Art

The term NO-synthase comprehends a family of enzymes that perform the enzymatic conversion of L-arginine to citrulline, during which reaction is produced a gaseous mediator with numerous functions, nitrogen monoxide, or NO.

NO-synthases exist in three forms, two constitutive forms, the nomenclature combining neuronal NO-synthase (or NOS 1) and endothelial NO-synthase (or NOS 3), and the inducible form (or NOS 2) (Medicine/Sciences, 1992, 8, pp. 843-845).

It is moreover understood in the present text that, unless otherwise indicated, the term “NO-synthase” covers all the isoforms of the enzyme.

Thus, according to the invention, the term “NO-synthase inhibitors” means any product that ultimately results in, irrespective of the isoform of NO-synthase, a reduction in the concentration of NO. Examples that may be mentioned include products which reduce the amount of active NO-synthase, which block the enzymatic activity of NO-synthase or its induction, or which inhibit the activity of the NO produced.

Nitrogen monoxide has, by virtue of its structure, an extra electron making it extremely chemically reactive. It is well known that such compounds are harmful and it is sought to optimally limit their production. Accordingly, in the case of nitrogen monoxide, NO-synthase inhibitors have been widely studied.

NO is a multifunctional signal molecule that is active in a wide variety of body tissues and systems. Besides its harmful effects on cells, associated with its hyperreactivity due to its structure comprising an extra electron, it is known, inter alia, as participating particularly in the cardiovascular system (blood pressure regulator with vasodilatory effect, platelet aggregation inhibitor with anticoagulant effect), in the nervous system (memory, modulation of the release of neurotransmitters), and in the immunological system (modulation of the immune defenses, inflammation, involvement in autoimmune pathologies).

It is now well accepted that NO plays a predominant role in the skin. NO may be synthesized by all the varieties of cells constituting the skin and, in this respect, it participates in numerous complex regulation processes such as regulating cell differentiation and/or proliferation, vasodilation, melanogenesis, and the response to environmental variations (homeostasis).

Its involvement in cell differentiation and proliferation (stimulatory effect), particularly for keratinocytes, associates it both with the growth of the epidermis and cicatrization and with hyperproliferative disorders (psoriasis).

As a result of its electronic hyperreactivity, which may lead to a degradation or even a destruction of cells, NO is involved in apoptotic processes and in intrinsic and/or extrinsic aging of the skin.

It participates in cutaneous inflammatory and immunological processes. Specifically, it is commonly accepted that NO plays a role in contact hypersensitivity reactions, in cutaneous allergic manifestations, and in the skin's immune response. Similarly, besides its direct proinflammatory role, it is the mediator between neuropeptides such as substance P and/or the peptide associated with the calcitonin gene (Calcitonin Gene Related Peptide, or CGRP) in cutaneous reaction processes of neurogenic origin, hence its involvement in “sensitive skin” phenomena. WO 97/15280 describes the advantage of using an NO-synthase inhibitor in the treatment of sensitive skin.

NO is also involved in reducing the skin's barrier effect and also in reducing skin moisturization.

The involvement of NO in vasodilation means that it is associated with cutaneous erythema, particularly erythema induced by ultraviolet radiation, localized or diffuse erythemal skin rashes, such as those caused by drugs, toxins and/or viral or bacterial infections, and rosacea.

NO is known as being an intermediate in melanogenesis induced by type B ultraviolet radiation (UVB). It is also thought to be one of the factors involved in disorders of hypermelanosis type.

Finally, NO appears to be involved in controlling sweating and also in hair loss.

The advantage of having available NO-synthase inhibitors may thus be appreciated. In this regard, many inhibitors have already been proposed in the prior art. Mention may be made more particularly of N^{G}-monomethyl-L-arginine (NMMA), the methyl ester of N^{G}-nitro-L-arginine (NAME), N^{G}-nitro-L-arginine (NNA), N^{G}-amino-L-arginine (NAA), N^{G},N^{G}-dimethyl-arginine (asymmetric dimethylarginine, known as ADMA), diphenyleneiodonium chloride, 2-(4-carboxyphenyl)-4,4,5,5-tetramethylimidazoline-1-oxy 3-oxide, 7-nitroindazole, N(5)-(1-iminoethyl)-L-ornithine, aminoguanidine, canavanine and ebselen.

Without questioning the efficacy of these products, it is noted that they are chemical compounds that can induce discomfort or even harmful side effects for the user, who generally prefers to use natural products. The aim of the present invention is to provide a novel NO-synthase inhibitor which furthermore is a natural NO-synthase inhibitor.

### SUMMARY OF THE INVENTION

It has now surprisingly and unexpectedly been determined that N,N′-bis(2-pyridyl)methyl-N,N′-bis(3,4,5-trimethoxybenzyl)ethylenediamine has the property of being an NO-synthase inhibitor, particularly of the inducible NO-synthase (NOS 2), which makes it a good candidate for use in applications in which it is found to be advantageous to employ an NO-synthase inhibitor, particularly in cosmetics.

N,N′-bis(2-pyridyl)methyl-N,N′-bis(3,4,5-trimethoxybenzyl) ethylenediamine is described for its use in pharmaceutical and cosmetic compositions for protecting the body against oxidative stress (EP 0 755 925).

To the knowledge of the present inventor, this compound has never been recognized as an NO-synthase inhibitor.

### DETAILED DESCRIPTION OF BEST MODE AND SPECIFIC/PREFERRED EMBODIMENTS OF THE INVENTION

The present invention thus features the administration of an effective amount of N,N′-bis(2-pyridyl)methyl-N,N′-bis(3,4,5-trimethoxybenzyl) ethylenediamine, in a physiologically acceptable medium, in a composition or for the preparation of a composition, said N,N′-bis(2-pyridyl)methyl-N,N′-bis(3,4,5-trimethoxybenzyl)ethylenediamine or composition comprised thereof being useful for inhibiting NO-synthase.

By the expression “physiologically acceptable medium” is intended a medium that is compatible with the skin, mucous membranes, the nails and the hair.

The present invention also features the administration of an effective amount of N,N′-bis(2-pyridyl)methyl-N,N′-bis(3,4,5-trimethoxybenzyl) ethylenediamine, in a physiologically acceptable medium, in a composition or for the preparation of a composition, said N,N′-bis(2-pyridyl)methyl-N,N′-bis (3,4,5-trimethoxybenzyl)ethylenediamine or composition comprised thereof being useful for application in any field in which an inhibition of NO-synthases is considered necessary, particularly in the field of skincare and/or haircare.

N,N′-bis(2-pyridyl)methyl-N,N′-bis(3,4,5-trimethoxybenzyl) ethylenediamine or composition comprised thereof may be used to slow down or even inhibit cell differentiation and/or proliferation, and/or vasodilation, and/or melanogenesis, and/or the response to environmental variations (homeostasis).

Thus, this invention also features administration of an effective amount of N,N′-bis(2-pyridyl)methyl-N,N′-bis(3,4,5-trimethoxybenzyl) ethylenediamine, in a physiologically acceptable medium, in a composition or for the preparation of a composition, the N,N′-bis(2-pyridyl)methyl-N,N′-bis (3,4,5-trimethoxybenzyl)ethylenediamine or composition comprised thereof being useful to slow down or even inhibit cell differentiation and/or proliferation, particularly to regulate the growth of the epidermis and/or to treat hyperproliferative disorders, for instance psoriasis.

This invention also features the administration of an effective amount of N,N′-bis(2-pyridyl)methyl-N,N′-bis(3,4,5-trimethoxybenzyl) ethylenediamine, in a physiologically acceptable medium, in a composition or for the preparation of a composition, the N,N′-bis(2-pyridyl)methyl-N,N′-bis(3,4,5-trimethoxybenzyl)ethylenediamine or composition comprised thereof being useful to inhibit the degradation and/or destruction of cells and to inhibit apoptotic processes, particularly of skin cells, most particularly of keratinocytes, and/or to treat the intrinsic and/or extrinsic aging of cells, particularly of skin cells.

The present invention also features the administration of an effective amount of N,N′-bis(2-pyridyl)methyl-N,N′-bis(3,4,5-trimethoxybenzyl) ethylenediamine, in a physiologically acceptable medium, in a composition or for the preparation of a composition, the N,N′-bis(2-pyridyl)methyl-N,N′-bis(3,4,5-trimethoxybenzyl)ethylenediamine or composition comprised thereof being useful to inhibit or even eliminate the symptoms associated with immunological and/or inflammatory phenomena associated with NO synthesis, for instance contact hypersensitivity reactions and/or allergic manifestations and/or the immune response, particularly as regards the skin.

The N,N′-bis(2-pyridyl)methyl-N,N′-bis(3,4,5-trimethoxybenzyl) ethylenediamine or composition comprised thereof is useful to reduce or even inhibit skin irritation caused by external agents. The skin irritant effect is a skin response usually reflected by redness, pain or stinging, this response being generated by chemical products of natural or synthetic origin applied topically to the skin. This irritation is accompanied by an impairment in epithelial function and/or structure, which is directly associated with the effect of the product of irritant nature.

The subject compound and compositions comprised thereof are therefore particularly suitable for treating skin reactions associated with processes of neurogenic origin such as certain forms of skin redness, and therefore for treating, reducing or eliminating the manifestations of “sensitive skin”. These are nonspecific reactions, which are distinguished from inflammation or allergy mechanisms. These symptoms are in particular subjective signs, which are essentially dysesthetic sensations. The term “dysesthetic sensations” means the more or less painful sensations experienced in an area of skin, for instance stinging, tingling, itching or pruritus, heating, discomfort, tautness, etc. Sensitive skin may be divided into two major clinical forms, irritable and/or reactive skin, and intolerant skin.

Irritable and/or reactive skin is skin that reacts with pruritus, i.e., with itching or stinging, to various factors such as the environment, emotions, foods, the wind, rubbing, shaving, soap, surfactants, hard water with a high calcium concentration, variations in temperature, or wool. In general, these signs are associated with dry skin with or without dry patches, or skin that shows noninflammatory erythema.

Intolerant skin is skin that reacts with sensations of heating, tautness, tingling and/or redness, to various factors such as the environment, emotions, foods and certain cosmetic products. In general, these signs are associated with hyperseborrheic or acneic skin with or without dry patches, and erythema.

“Sensitive” scalps have a more unequivocal clinical semiology: the sensations of pruritus and/or stinging and/or heating are essentially triggered by local factors such as rubbing, soap, surfactants, hard water with a high calcium concentration, shampoos or lotions. These sensations are also occasionally triggered by factors such as the environment, emotions and/or foods. Erythema and hyperseborrhea of the scalp, and a dandruff-infested state, are frequently associated with the above signs.

This invention also features the administration of an effective amount of N,N′-bis(2-pyridyl)methyl-N,N′-bis(3,4,5-trimethoxybenzyl) ethylenediamine, in a physiologically acceptable medium, in a composition or for the preparation of a composition, the N,N′-bis(2-pyridyl)-methyl-N,N′-bis(3,4,5-trimethoxybenzyl)ethylenediamine or composition comprised thereof being useful to increase the skin's barrier effect or moisturization of the skin.

This invention also features the administration of an effective amount of N,N′-bis(2-pyridyl)methyl-N,N′-bis(3,4,5-trimethoxybenzyl) ethylenediamine, in a physiologically acceptable medium, in a composition or for the preparation of a composition, the N,N′-bis(2-pyridyl)methyl-N,N′-bis(3,4,5-trimethoxybenzyl)ethylenediamine or composition comprised thereof being useful to treat rosacea and/or skin erythema, particularly erythema induced by ultraviolet radiation, and/or localized or diffuse erythemal skin rashes such as those caused by drugs, toxins and/or viral or bacterial infections.

The present invention also features the administration of an effective amount of N,N′-bis(2-pyridyl)methyl-N,N′-bis(3,4,5-trimethoxybenzyl)ethylenediamine, in a physiologically acceptable medium, in a composition or for the preparation of a composition, the N,N′-bis(2-pyridyl) methyl-N,N′-bis(3,4,5-trimethoxybenzyl)ethylenediamine or composition comprised thereof being useful to inhibit melanogenesis induced by type A and/or B ultraviolet radiation, and/or to treat disorders of hypermelanosis type.

The present invention also features the administration of an effective amount of N,N′-bis(2-pyridyl)methyl-N,N′-bis(3,4,5-trimethoxybenzyl)ethylenediamine, in a physiologically acceptable medium, in a composition or for the preparation of a composition, the N,N′-bis(2-pyridyl) methyl-N,N′-bis(3,4,5-trimethoxybenzyl)ethylenediamine or composition comprised thereof being useful to control sweating and/or to stimulate lipolysis or to reduce or inhibit hair loss.

According to the invention, the composition comprising N,N′-bis(2-pyridyl)methyl-N,N′-bis(3,4,5-trimethoxybenzyl)ethylenediamine may be a cosmetic or dermatological composition. Preferably, according to the invention, the composition is a cosmetic composition.

Preferably, according to the invention, N,N′-bis(2-pyridyl)methyl-N,N′-bis(3,4,5-trimethoxybenzyl)ethylenediamine or the composition comprised thereof is applied topically to the skin of an individual in need of such treatment.

According to the invention, the amount of N,N′-bis(2-pyridyl) methyl-N,N′-bis(3,4,5-trimethoxybenzyl)ethylenediamine extract used in the composition obviously depends on the desired effect and may thus vary within a wide range.

To provide an order of magnitude, according to the invention, N,N′-bis(2-pyridyl)methyl-N,N′-bis(3,4,5-trimethoxybenzyl)ethylenediamine may be used in an amount representing from 10-4% to 20% of the total weight of the composition, and preferably in an amount representing from 5×10-3% to 10% of the total weight of the composition.

Needless to say, according to the invention, the N,N′-bis(2-pyridyl)methyl-N,N′-bis(3,4,5-trimethoxybenzyl)ethylenediamine may be combined with other NO-synthase inhibitors, such as plant extracts, for instance an extract of at least one plant of the species Olea europaea or an extract of Ginkgo biloba or an extract of Vitis vinifera, or alternatively an extract of green tea or of cacao.

This invention also features a cosmetic treatment process for treating disorders associated with NO synthesis, wherein a cosmetic composition comprising at least N,N′-bis(2-pyridyl)methyl-N,N′-bis(3,4,5-trimethoxybenzyl)ethylenediamine in a physiologically acceptable medium is topically applied onto the skin, the hair and/or mucous membranes.

The cosmetic treatment process of the invention is directed toward improving the appearance of the individual suffering from disorders caused by NO synthesis.

The cosmetic treatment process of the invention may be performed especially by applying the cosmetic compositions as defined above, according to the usual technique for using these compositions. Thus, for example, it is possible to apply creams, gels, sera, lotions, makeup-removing milks or antisun compositions to the skin or to dry hair, to apply a hair lotion to wet hair, or shampoos, or alternatively to apply toothpaste to the gums.

Irrespective of the form of the composition according to the invention in which N,N′-bis(2-pyridyl)methyl-N,N′-bis(3,4,5-trimethoxybenzyl)ethylenediamine is formulated, this composition may be ingested, injected or applied to the skin (to any area of body skin), the hair, the nails or mucous membranes (buccal, jugal, gingival, genital or conjunctival mucosa). Depending on the mode of administration, the composition according to the invention may be in any presentation form normally used.

For topical application to the skin, the composition may especially be in the form of an aqueous or oily solution or a dispersion, of the lotion or serum type, emulsions of liquid or semi-liquid consistency of the milk type, obtained by dispersing a fatty phase in an aqueous phase (O/W) or conversely (W/O), or suspensions or emulsions of soft consistency of the aqueous or anhydrous cream or gel type, or alternatively microcapsules or microparticles or vesicular dispersions of ionic and/or nonionic type. These compositions are prepared according to the usual methods.

They may also be used for the hair in the form of aqueous, alcoholic or aqueous-alcoholic solutions, or in the form of creams, gels, emulsions or mousses, or alternatively in the form of aerosol compositions also comprising a pressurized propellant.

For injection, the composition may be in the form of an aqueous or oily lotion or in the form of a serum. For the eyes, it may be in the form of drops, and for ingestion, it may be in the form of capsules, granules, syrups or tablets.

The amounts of the various constituents of the compositions that may be used according to the invention are those that are conventionally used in the fields under consideration.

These compositions especially constitute cleansing, protective, treating or care creams for the face, for the hands, for the feet, for the major anatomical folds or for the body (for example day creams, night creams, makeup-removing creams, foundation creams or antisun creams), fluid foundations, makeup-removing milks, protective or care body milks, antisun milks, skincare lotions, gels or mousses, for instance cleansing lotions, antisun lotions or artificial tanning lotions, bath compositions, deodorizing compositions comprising a bactericidal agent, aftershave gels or lotions, hair-removing creams, compositions for treating insect bites, pain-relief compositions and compositions for treating certain skin diseases, for instance eczema, rosacea, psoriasis, lichens and severe pruritus.

The compositions according to the invention may also consist of solid preparations constituting cleansing bars or soaps.

The compositions may also be packaged in the form of an aerosol composition also comprising a pressurized propellant.

The composition according to the invention may also be a haircare composition, and especially a shampoo, a hairsetting lotion, a treating lotion, a styling cream or gel, a dye composition (especially oxidation dyes) optionally in the form of coloring shampoos, restructuring lotions for the hair, a permanent-waving composition (especially a composition for the first stage of a permanent-waving operation), a lotion or gel for preventing hair loss, an antiparasitic shampoo, etc.

The composition may also be for buccodental use, for example a toothpaste. In this case, the composition may contain adjuvants and additives that are common for compositions for buccal use, and especially surfactants, thickeners, humectants, polishing agents such as silica, various active ingredients, for instance fluorides, in particular sodium fluoride, and optionally sweeteners, for instance sodium saccharinate.

When the composition is an emulsion, the proportion of the fatty phase may range from 5% to 80% by weight and preferably from 5% to 50% by weight relative to the total weight of the composition. The oils, waxes, emulsifiers and coemulsifiers used in the composition in emulsion form are chosen from those conventionally used in the cosmetics field. The emulsifier and coemulsifier are present in the composition in a proportion ranging from 0.3% to 30% by weight and preferably from 0.5% to 20% by weight relative to the total weight of the composition. The emulsion may also contain lipid vesicles.

When the composition is an oily solution or gel, the fatty phase may represent more than 90% of the total weight of the composition.

In a known manner, the cosmetic composition may also contain adjuvants that are common in cosmetics, such as hydrophilic or lipophilic gelling agents, hydrophilic or lipophilic additives, preservatives, antioxidants, solvents, fragrances, fillers, screening agents, odor absorbers and dyestuffs and colorants. The amounts of these various adjuvants are those conventionally used in cosmetics, for example from 0.01% to 10% of the total weight of the composition. Depending on their nature, these adjuvants may be introduced into the fatty phase, into the aqueous phase and/or into lipid spherules.

As oils or waxes that may be used in the invention, mention may be made of mineral oils (liquid petroleum jelly), plant oils (liquid fraction of shea butter, sunflower oil), animal oils (perhydrosqualene), synthetic oils (purcellin oil), silicone oils or waxes (cyclomethicone), fluoro oils (perfluoropolyethers), beeswax, carnauba wax or paraffin wax. Fatty alcohols and fatty acids (stearic acid) may be added to these oils.

As examples of emulsifiers that may be used in the invention, mention may be made of glyceryl stearate, polysorbate 60 and the mixture of PEG-6/PEG-32/glycol stearate sold under the name Tefose® 63 by the company Gattefosse.

As solvents that may be used in the invention, mention may be made of lower alcohols, especially ethanol and isopropanol, and propylene glycol.

As hydrophilic gelling agents that may be used in the invention, mention may be made of carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, mention may be made of modified clays, for instance bentones, metal salts of fatty acids, for instance aluminum stearates, hydrophobic silica, ethylcellulose and polyethylene.

The composition may contain other hydrophilic active agents, for instance proteins or protein hydrolyzates, amino acids, polyols, urea, allantoin, sugars and sugar derivatives, water-soluble vitamins, plant extracts and hydroxy acids.

Lipophilic active agents that may be used include retinol (vitamin A) and its derivatives, tocopherol (vitamin E) and its derivatives, essential fatty acids, ceramides, essential oils and salicylic acid and its derivatives.

According to the invention, the composition may combine at least one N,N′-bis(2-pyridyl)methyl-N,N′-bis(3,4,5-trimethoxybenzyl)ethylenediamine extract with other active agents intended especially for preventing and/or treating skin complaints, conditions or afflictions. Examples of these active agents that may be mentioned include:

agents for modifying cutaneous differentiation and/or proliferation and/or pigmentation, such as retinoic acid and its isomers, retinol and its esters, vitamin D and its derivatives, kojic acid or hydroquinone;

antibacterial agents such as clindamycin phosphate, erythromycin or antibiotics of the tetracycline class;

antiparasitic agents, in particular metronidazole, crotamiton or pyrethroids;

antifungal agents, in particular compounds belonging to the imidazole class, such as econazole, ketoconazole or miconazole or the salts thereof, polyene compounds, such as amphotericin B, compounds of the allylamine family, such as terbinafine, or octopirox;

nonsteroidal antiinflammatory agents, for instance ibuprofen and its salts, diclofenac and its salts, acetylsalicylic acid, acetaminophen or glycyrrhetinic acid;

anesthetics such as lidocaine hydrochloride and its derivatives;

antipruriginous agents, for instance thenaldine, trimeprazine or cyproheptadine;

keratolytic agents such as α- and β-hydroxycarboxylic acids or β-keto carboxylic acids, and the salts, amides or esters thereof and more particularly hydroxy acids such as glycolic acid, lactic acid, salicylic acid, citric acid and fruit acids in general, and 5-n-octanoylsalicylic acid;

free-radical scavengers, such as α-tocopherol or its esters, superoxide dismutases, certain metal-chelating agents or ascorbic acid and its esters;

antiseborrheic agents such as progesterone;

antidandruff agents, for instance octopirox or zinc pyrithione;

antiacne agents, for instance retinoic acid or benzoyl peroxide;

extracts of plant or microbial origin;

peptides and derivatives thereof, for instance the tripeptide Lys-Pro-Val.

In order to further illustrate the present invention and the advantages thereof, the following specific examples/compositions are given, it being understood that same are intended only as illustrative and in nowise limitative.

In said examples/compositions to follow, all parts and percentages are given by weight.

### EXAMPLE 1

### Biological Activity of N,N′-bis(2-pyridyl)methyl-N,N′-bis (3,4,5-trimethoxybenzyl)ethylenediamine

The activity of N,N′-bis(2-pyridyl)methyl-N,N′-bis(3,4,5-trimethoxybenzyl)ethylenediamine on inducible NO-synthase was evaluated in the test described by Heck et al., (J.B.C., Vol. 267, No. 30, 21277-21280, Oct. 25, 1992).

The object of this test is to show the reduction in the concentration of nitrate and nitrite, ultimately, after stimulating NO-synthase 2.

The following controls were introduced into the tests:

A: positive control (induction of the enzyme): mixtures of interferon-(1000 U/ml) and of interleukin 1-(100 U/ml);

B: negative control (maximum inhibition): N^{G}-monomethyl-L-arginine (L form) at 200 μm;

C: control of inhibition specificity: N^{G}-monomethyl-L-arginine (D form) at 200 μm.

To determine the activity of the test product, the amount of stable NO reaction products (nitrites and nitrates) is measured using the “nitric calorimetric assay” kit marketed by Boehringer under the reference 1756.28.

N,N′-bis(2-pyridyl)methyl-N,N′-bis(3,4,5-trimethoxybenzyl) ethylenediamine was tested at concentrations of 10 μM, 50 μM and 100 μM in ethanol.

| Test Product | % Inhibition |
|---|---|
| A | 0 |
| B | 100 |
| C | 0 |
| N,N′-bis(2-pyridyl)methyl-N,N′-bis(3, | 18.8% |
| 4,5-trimethoxybenzyl)ethylenediamine: | |
| 10 μM | |
| N,N′-bis(2-pyridyl)methyl-N,N′-bis(3, | 48.3% |
| 4,5-trimethoxybenzyl)ethylenediamine: | |
| 50 μM | |
| N,N′-bis(2-pyridyl)methyl-N,N′-bis(3, | 62% |
| 4,5-trimethoxybenzyl)ethylenediamine: | |
| 100 μM | |

N,N′-bis(2-pyridyl)methyl-N,N′-bis(3,4,5-trimethoxybenzyl) ethylenediamine has an inhibitory effect on inducible NO-synthase.

### EXAMPLE 2

The following are examples of specific formulations illustrating the invention. These compositions were formulated by simple mixing of the various components.

Composition 1: Facial gel

| | |
|---|---|
| Compound of invention | 0.1% |
| Methyl paraben | 0.2% |
| Carbomer | 0.7% |
| Polyethylene glycol (8 EO) | 10.0% |
| Imidazolidinylurea | 0.3% |
| Triethanolamine | 0.58% |
| Water qs | 100% |

Composition 2: Lotion

| | |
|---|---|
| Compound of invention | 2.00% |
| Antioxidant | 0.05% |
| Isopropanol | 40.00% |
| Preservative | 0.30% |
| Water qs | 100% |

Composition 3: Care gel

| | |
|---|---|
| Compound of invention | 2.00% |
| Hydroxypropylcellulose* | 1.00% |
| Antioxidant | 0.05% |
| Isopropanol | 40.00% |
| Preservative | 0.30% |
| Water qs | 100% |

Composition 4: Care cream (oil-in-water emulsion)

| | |
|---|---|
| Compound of invention | 5.00% |
| Glyceryl stearate | 2.00% |
| Polysorbate 60** | 1.00% |
| Stearic acid | 1.40% |
| Triethanolamine | 0.70% |
| Carbomer | 0.40% |
| Liquid fraction of shea butter | 12.00% |
| Perhydrosqualene | 12.00% |
| Antioxidant | 0.05% |
| Fragrance | 0.50% |
| Preservative | 0.30% |
| Water qs | 100% |

Composition 5: Shampoo

| | |
|---|---|
| Compound of invention | 0.50% |
| Hydroxypropylcellulose* | 1.00% |
| Fragrance | 0.50% |
| Preservative | 0.30% |
| Water qs | 100% |

Composition 6: Care cream (oil/water emulsion)

| | |
|---|---|
| Compound of invention | 5.00% |
| Glyceryl stearate | 2.00% |
| Polysorbate 60** | 1.00% |
| Stearic acid | 1.40% |
| 5-n-Octanoylsalicylic acid | 0.50% |
| Triethanolamine | 0.70% |
| Carbomer | 0.40% |
| Liquid fraction of shea butter | 12.00% |
| Perhydrosqualene | 12.00% |
| Antioxidant | 0.05% |
| Fragrance | 0.50% |
| Preservative | 0.30% |
| Water qs | 100% |

Composition 7: Pain-relief gel

| | |
|---|---|
| Compound of invention | 3.00% |
| Hydroxypropylcellulose* | 1.00% |
| Antioxidant | 0.05% |
| Lidocaine hydrochloride | 2.00% |
| Isopropanol | 40.00% |
| Preservative | 0.30% |
| Water qs | 100% |

Composition 8: Care cream for solar erythema (oil-in-water emulsion)

| | |
|---|---|
| Compound of invention | 5.00% |
| Glyceryl stearate | 2.00% |
| Polysorbate 60** | 1.00% |
| Stearic acid | 1.40% |
| Glycyrrhetinic acid | 2.00% |
| Triethanolamine | 0.70% |
| Carbomer | 0.40% |
| Liquid fraction of shea butter | 12.00% |
| Sunflower oil | 10.00% |
| Antioxidant | 0.05% |
| Fragrance | 0.50% |
| Preservative | 0.30% |
| Water qs | 100% |

Composition 9: Gel for treating acne

| | |
|---|---|
| Compound of invention | 5.00% |
| All-trans-retinoic acid | 0.05% |
| Hydroxypropylcellulose* | 1.00% |
| Antioxidant | 0.05% |
| Isopropanol | 40.00% |
| Preservative | 0.30% |
| Water qs | 100% |

Composition 10: Lotion for removing acne scars

| | |
|---|---|
| Compound of invention | 5.00% |
| Glycolic acid | 50.00% |
| Hydroxypropylcellulose* | 0.05% |
| Preservative | 0.30% |
| NaOH | qs pH = 2.8 |
| Ethanol | qs 100% |

*Klucel H ® marketed by Hercules
**Tween 60 ® marketed by ICI

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for inhibiting NO-synthase, comprising administering to an individual in need of such treatment, a thus effective amount of N,N′-bis(2-pyridyl)methyl-N,N′-bis(3,4,5-trimethoxybenzyl)ethylenediamine or therapeutic/cosmetic composition comprised thereof, formulated into a physiologically acceptable medium therefor.

2. A regime or regimen for slowing or inhibiting cell differentiation and/or proliferation, comprising administering to an individual in need of such treatment, a thus effective NO-synthase inhibiting amount of N,N′-bis(2-pyridyl)methyl-N,N′-bis(3,4,5-trimethoxybenzyl)ethylenediamine or therapeutic/cosmetic composition comprised thereof, formulated into a physiologically acceptable medium therefor.

3. A regime or regimen for slowing or inhibiting the growth of the epidermis and/or for treating hyperproliferation disorders, comprising administering to an individual in need of such treatment, a thus effective NO-synthase inhibiting amount of N,N′-bis(2-pyridyl)methyl-N,N′-bis(3,4,5-trimethoxybenzyl) ethylenediamine, or therapeutic/cosmetic composition comprised thereof, formulated into a physiologically acceptable medium therefor.

4. A regime or regimen for inhibiting the degradation and/or destruction of cells, comprising administering to an individual in need of such treatment, a thus effective NO-synthase inhibiting amount of N,N′-bis(2-pyridyl)methyl-N,N′-bis (3,4,5-trimethoxybenzyl)ethylenediamine, or therapeutic/cosmetic composition comprised thereof, formulated into a physiologically acceptable medium therefor.

5. A regime or regimen for inhibiting a cellular apoptotic process, comprising administering to an individual in need of such treatment, a thus effective NO-synthase inhibiting amount of N,N′-bis(2-pyridyl)methyl-N,N′-bis(3,4,5-trimethoxybenzyl)ethylenediamine, or therapeutic/cosmetic composition comprised thereof, formulated into a physiologically acceptable medium therefor.

6. A regime or regimen for treating intrinsic and/or extrinsic aging, comprising administering to an individual in need of such treatment, a thus effective NO-synthase inhibiting amount of N,N′-bis(2-pyridyl)methyl-N,N′-bis (3,4,5-trimethoxybenzyl)ethylenediamine, or therapeutic/cosmetic composition comprised thereof, formulated into a physiologically acceptable medium therefor.

7. A regime or regimen for inhibiting or suppressing an immunological and/or inflammatory process, comprising administering to an individual in need of such treatment, a thus effective NO-synthase inhibiting amount of N,N′-bis(2-pyridyl)methyl-N,N′-bis(3,4,5-trimethoxybenzyl)ethylenediamine, or therapeutic/cosmetic composition comprised thereof, formulated into a physiologically acceptable medium therefor.

8. A regime or regimen for treating a contact hypersensitivity reaction and/or an allergic manifestation and/or an immune response, comprising administering to an individual in need of such treatment, a thus effective NO-synthase inhibiting amount of N,N′-bis(2-pyridyl)methyl-N,N′-bis(3,4,5-trimethoxybenzyl) ethylenediamine, or therapeutic/cosmetic composition comprised thereof, formulated into a physiologically acceptable medium therefor.

9. A regime or regimen for treating a skin reaction neurogenic in origin, comprising administering to an individual in need of such treatment, a thus effective NO-synthase inhibiting amount of N,N′-bis(2-pyridyl)methyl-N,N′-bis (3,4,5-trimethoxybenzyl)ethylenediamine, or therapeutic/cosmetic composition comprised thereof, formulated into a physiologically acceptable medium therefor.

10. A regime or regimen for treating “sensitive skin,” comprising administering to an individual in need of such treatment, a thus effective NO-synthase inhibiting amount of N,N′-bis(2-pyridyl)methyl-N,N′-bis(3,4,5-trimethoxybenzyl)ethylenediamine, or therapeutic/cosmetic composition comprised thereof, formulated into a physiologically acceptable medium therefor.

11. A regime or regimen for increasing the barrier effect or moisturization of the skin, comprising administering to an individual in need of such treatment, a thus effective NO-synthase inhibiting amount of N,N′-bis(2-pyridyl)methyl-N,N′-bis(3,4,5-trimethoxybenzyl)ethylenediamine, or therapeutic/cosmetic composition comprised thereof, formulated into a physiologically acceptable medium therefor.

12. A regime or regimen for treating erythema, comprising administering to an individual in need of such treatment, a thus effective NO-synthase inhibiting amount of N,N′-bis(2-pyridyl)methyl-N,N′-bis(3,4,5-trimethoxybenzyl) ethylenediamine, or therapeutic/cosmetic composition comprised thereof, formulated into a physiologically acceptable medium therefor.

13. A regime or regimen for treating a localized or diffuse erythemal skin rash, comprising administering to an individual in need of such treatment, a thus effective NO-synthase inhibiting amount of N,N′-bis(2-pyridyl)methyl-N,N′-bis (3,4,5-trimethoxybenzyl)ethylenediamine, or therapeutic/cosmetic composition comprised thereof, formulated into a physiologically acceptable medium therefor.

14. A regime or regimen for treating rosacea, comprising administering to an individual in need of such treatment, a thus effective NO-synthase inhibiting amount of N,N′-bis(2-pyridyl)methyl-N,N′-bis(3,4,5-trimethoxybenzyl) ethylenediamine, or therapeutic/cosmetic composition comprised thereof, formulated into a physiologically acceptable-medium therefor.

15. A regime or regimen for inhibiting melanogenesis induces by UV-A and/or UV-B radiation and/or for treating a hypermelanosis disorder, comprising administering to an individual in need of such treatment, a thus effective NO-synthase inhibiting amount of N,N′-bis(2-pyridyl)methyl-N,N′-bis(3,4,5-trimethoxybenzyl)ethylenediamine, or therapeutic/cosmetic composition comprised thereof, formulated into a physiologically acceptable medium therefor.

16. A regime or regimen for controlling sweating, comprising administering to an individual in need of such treatment, a thus effective NO-synthase inhibiting amount of N,N′-bis(2-pyridyl)methyl-N,N′-bis(3,4,5-trimethoxybenzyl) ethylenediamine, or therapeutic/cosmetic composition comprised thereof, formulated into a physiologically acceptable medium therefor.

17. A regime or regimen for inhibiting hair loss, comprising administering to an individual in need of such treatment, a thus effective NO-synthase inhibiting amount of N,N′-bis(2-pyridyl)methyl-N,N′-bis(3,4,5-trimethoxybenzyl) ethylenediamine, or therapeutic/cosmetic composition comprised thereof, formulated into a physiologically acceptable medium therefor

18. The regime or regimen as defined by claim 1, comprising administering a therapeutic/cosmetic composition which comprises from 10⁻⁴% to 20% by weight of said N,N′-bis(2-pyridyl)methyl-N,N′-bis(3,4,5-trimethoxybenzyl) ethylenediamine.

19. The regime or regimen as defined by claim 1, comprising administering a therapeutic/cosmetic composition which comprises from 5×10⁻³% to 10% by weight of said N,N′-bis(2-pyridyl)methyl-N,N′-bis(3,4,5-trimethoxybenzyl) ethylenediamine.

20. A cosmetic regime or regimen for treating a disorder associated with NO-synthesis, comprising topically applying onto the skin, the hair and/or the mucous membranes of an individual in need of such treatment, a thus effective amount of N,N′-bis(2-pyridyl)methyl-N,N′-bis(3,4,5-trimethoxybenzyl)ethylenediamine formulated into a topically applicable, physiologically acceptable medium therefor.

21. A therapeutic/cosmetic composition suited for treating a disorder associated with NO-synthesis, comprising a thus effective amount of N,N′-bis(2-pyridyl)methyl-N,N′-bis(3,4,5-trimethoxybenzyl)ethylenediamine formulated into a physiologically acceptable medium therefor and constituting a cream, a gel, a serum, a lotion, a milk, a sunscreen, an emulsion, a mousse, an aerosol, a shampoo, a cleaning bar, a soap, a toothpaste, a syrup, capsules, tablets, granules, microcapsules, microparticles, or a vesicular dispersion.

22. The therapeutic/cosmetic composition as defined by claim 21, further comprising at least one other NO-synthase inhibitor.

23. The therapeutic/cosmetic composition as defined by claim 21, further comprising at least one other active agent selected from the group consisting of an agent for modifying cutaneous differentiation and/or proliferation and/or pigmentation; an antibacterial agent; an antiparasitic agent; and antifungal agent; a nonsteroidal anti-inflammatory agent; a keratolytic agent; a free-radical scavenger; an antiseborrheic agent; an antidandruff agent; an antiacne agent; an extract of plant or microbial origin; and a peptide or derivative thereof.
